(19) United States
(12) Patent Application Publication
(10) Pub. No.: US 2006/0019348 A1
(43) Pub. Date: Jan. 26, 2006

(54) Method of mass producing silk protein and gene recombinant silk-like protein with added functionality

(76) Inventor: Tetsuo Asakura, Tokyo (JP)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/197,315

(22) Filed: Aug. 5, 2005

Related U.S. Application Data
(63) Continuation-in-part of application No. 10/515,264.

(51) Int. Cl. C12P 21/06; C12N 15/74; C12N 1/21; C07K 14/435

(52) U.S. Cl. 435/69.1; 530/353; 435/252.33; 435/488

(57) ABSTRACT

A method of producing silk or silk-like protein wherein silk or a silk-like polymer comprising at least one protein selected from among domestic silkworm fibroin, wild silkworm fibroin, elastin and fibronectin, and essentially comprising the aforesaid domesticated silkworm fibroin or silkworm fibroin is designed. The minimum unit of the silk or the thus designed polymer is synthesized, and the polymer of the minimum unit thus synthesized is integrated into at least one expression vector selected from among expression vectors containing T7 promoter. Then, the expression vector is integrated into E. coli BL21 (DE3) pLysS or BLR(DE3) pLysS, and the E. coli is grown in a medium selected from among composite media.

### RELATED APPLICATION

This application is a continuation-in-part of U.S. Ser. No. 10/515,264, filed Nov. 22, 2004 which is hereby incorporated by reference in its entirety.

### FIELD OF THE INVENTION

This invention relates to a method for mass production of silk protein and gene recombinant silk-like protein with added functionality, and in particular, a method of mass producing gene recombinant silk-like protein with imparted cellular adhesiveness, elasticity or hardness.

### BACKGROUND ART

Silk is a high strength, high elasticity fiber, and since it has aminoacids contained in living organisms as its structural unit, it is biocompatible and is used in various fields such as clothing, food and cosmetics.

A detailed structural analysis of silk has been made in order to explore the origin of these outstanding physical properties. The primary structure of silk is a block copolymer in which some kinds of regular aminoacid sequences (motifs) are repeated about ten times. Recently, now that the secondary structure of these motifs has been clarified, more information is coming to light regarding the correlation between the structure of silk fiber and its physical properties.

In recent years, research has been carried out on artificially synthesizing silk-like protein from synthetic DNA which codes for silk protein. Since silk protein has a repetitive structure of identical aminoacid sequences, the number of times a specific aminoacid appears, increases. Therefore, if some types of aminoacyl-tRNAs are depleted, there may be an error at the conclusion of protein synthesis. Moreover, since repetition of DNA sequences may lead to sequence recombination within E. coli, it was still difficult to obtain silk-like protein having a repetition sequence in large amounts by using E. coli.

In order to increase the expression efficiency, a vector which combines a powerful T7 promoter with an expression vector came into wide use, but there was the disadvantage that if the target protein was too stressful for E. coli on account of its powerful action, this protein affected the growth of the bacteria and the expression efficiency did not increase.

The Inventor therefore arbitrarily selected domesticated silkworm silk fibroin, wild silkworm silk fibroin, elastin and the functional motif of fibronectin to design four functional silk-like proteins. It was then found that, in the case of functional silk-like proteins with these four repetition sequences, by optimizing selection of expression vectors, host E. coli and expression conditions, it was possible to mass produce the functional silk-like proteins by using E. coli, and it was possible to apply this procedure also to common silk protein, which led to the present invention.

It is therefore an object of this invention to provide a procedure for mass production of silk protein and silk-like protein with functional properties.

### SUMMARY OF THE INVENTION

According to this invention, a silk-like protein is produced by selecting one of domesticated silkworm silk fibroin, wild silkworm silk fibroin, elastin and fibronectin to design a silk-like polymer comprising a combination of two or more proteins requiring one of domesticated silkworm silk fibroin or wild silkworm silk fibroin, synthesizing the designed minimum unit of the polymer, and integrating the polymer having this synthesized minimum unit into at least one expression vector selected from expression vectors including T7 promoter. Subsequently, this expression vector was integrated into one of the E. coli BL21(DE3) pLysS and BLR(DE3) pLysS, and the E. coli was grown using a culture medium selected from composite culture media.

According to this invention, it is preferred to lower the temperature to 2-7° C. below the optimum growth temperature of E. coli, and in particular it is preferred to use an expression vector which contains a T71ac promoter as the expression vector which contains the T7 promoter.

### BRIEF DESCRIPTION OF THE DRAWINGS

FIG. 1 is the result of SDS-PAGE (sodium-dodecyl-sulfate-polyacrylamide gel electrophoresis) when SLP 2, 4, 6 are expressed in the E. coli strain BL21 (DE3) pLysS.

FIG. 2 is the result of detection by Western blot using His-Tag antibody, after separating SLPA4 by SDS-PAGE.

FIG. 3 is the result of detection by Western blot using His-Tag antibody, after separating SELP8 by SDS-PAGE.

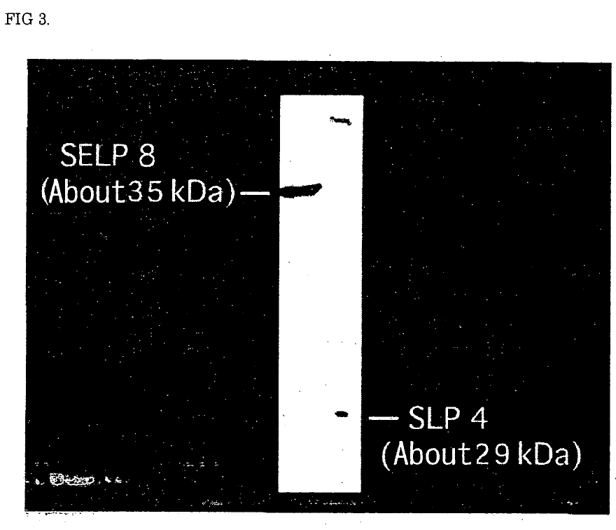

FIG. 4 is the result of detection by Western blot using His-Tag antibody, after separating SLPF5 by SDS-PAGE.

### DETAILED DESCRIPTION OF THE INVENTION

In this invention, domesticated silkworm silk fibroin means the protein secreted from the posterior silk gland of a domesticated silkworm (Bombyx mori), and wild silkworm silk fibroin means the protein secreted from the posterior silk gland of a wild silkworm.

These are described in the annals of the Silk Yarn Dictionary, Japanese Society of Sericultural Science (1979).

Elastin is a protein which is responsible for elasticity in the tissues of various organisms. In the primary structure of this elastin, there are regions where multiple sequences of 5 aminoacid residues, Val-Pro-Gly-Val-Gly (SEQ ID NO: 1), occur with high frequency (e.g., for chick elastin, Bressan, G. M., Argos, P. and Stanley, K. K., Repeating structure of chick tropoelastin revealed by complementary DNA cloning, Biochemistry 26, 1497-1503 (1987), and for bovine elastin, Raju, K and Anwar, R. A. Primary structures of bovine elastin a, b and c deduced from the sequences of cDNA clones, J. Biol. Chem. 262, 5755-5762 (1987)). Therefore, the functional motif of the elastin in this invention means the aminoacid sequence of the above-mentioned 5 residues.

Fibronectin is a protein having cellular adhesiveness which exists in the extra-cellular matrix of various organisms, and this cellular adhesiveness is related to an aminoacid sequence of 4 residues, i.e., Arg-Gly-Asp-Ser (SEQ ID NO: 2) contained therein (Reference: Pierschbacher MD, Rouslahti E, Nature 30930-33 (1984)). The aminoacid sequence of the 4 residues Arg-Gly-Asp-Ser (SEQ ID NO: 2) contained in fibronectin is the functional motif of fibronectin. Hence, in this invention, to support the secondary structure required for Arg-Gly-Asp-Ser (SEQ ID NO: 2) to express cellular adhesiveness, Thr-Gly-Arg-Gly-Asp-Ser-Pro-Ala (SEQ ID NO: 3) was used as the functional motif. Therefore, although this sequence of 8 residues is not limiting, the aforesaid extra part of the sequence was adopted as the aminoacid sequence surrounding the Arg-Gly-Asp-Ser (SEQ ID NO: 2) sequence in human fibronectin due to considerations of biocompatibility in case it is used as artificial skin.

According to this invention, the protein is designed based on the view, proposed by Lewis et al, that the physical properties and functionality of silk vary depending on the types of motif contained in silk protein, and their combinations. In this invention, the motifs contained in natural silk fibers, and the motif sequences considered to express functions specific to elastin and fibronectin (i.e., heat response (condensation when temperature is increased which stops dissolution in water), and cellular adhesiveness)), can be combined in various ways.

Specifically, to design a protein with novel physical properties and functionality not present in natural fibers, SLP (silk-like protein), SLPA (silk-like protein with polyalanine), SELP (silk and elastin-like protein), and SLPF (silk-like protein with fibronectin), were designed by variously rearranging the motifs in natural silk fiber, as follows.

SLP (Silk-Like Protein):

Combination of the aminoacid sequence in domesticated silkworm silk (GlyAlaGlySerGlyAla)₃ (SEQ ID NO: 4) and the aminoacid sequence GlyGlyAlaGlySerGlyTyrGlyGlyGlyTyrGlyHisGlyTyrGly SerAspGlyGly (SEQ ID NO: 5) of the glycine-rich region in wild silkworm silk.

SLPA (Silk-like Protein with Polyalanine): Combination of the aminoacid sequences GlyValGlyAlaGlyTyr (SEQ ID NO: 6), GlyAlaGlyAlaGlyTyr (SEQ ID NO: 7), GlyValGlyAlaGlyTyr (SEQ ID NO: 6) and GlyAlaGlyValGlyTyr (SEQ ID NO: 8) in domesticated silkworm silk, and the aminoacid sequence (A)₁₈ (SEQ ID NO: 9) similar to the polyalanine region in wild silkworm silk.

SELP (Silk and Elastin-Like Protein):

Combination of the aminoacid sequence (GlyAlaGlySerGlyAla)₃ (SEQ ID NO: 4) in domesticated silkworm silk, and the amino acid sequence (GlyValProGlyVal)₂ (SEQ ID NO: 10) in elastin.

SLPF: (Silk-like Protein with Fibronectine) Combination of the aminoacid sequence (GlyAlaGlySerGlyAla)₃ (SEQ ID NO: 4) in domesticated silkworm silk and the aminoacid sequence ThrGlyArgGlyAspSerProAla (SEQ ID NO: 11) in fibronectin.

The fibers must contain a crystalline region and an amorphous region, and when a new silk-like protein is designed, the motifs must be combined so that these regions are formed simultaneously. For example, in SLP and SLPA, in domesticated silkworm silk and silk from the Eri silkworm, which is a kind of wild silkworm, motifs which form crystalline regions or amorphous regions are respectively combined. In the case of SELP and SLPF, in addition to thermal stability and biodegradability, still more functions can be imparted by combining the functional motifs of elastin and fibronectin with silk protein for use not only as a fiber but also as a biopolymer.

In this invention, pET30a which contains T7 promoter as an expression vector, and the expression inductor BL21 (DE3)pLysS or BLR (DE3)pLysS as the host E. coli used for expression, are selected. Due to these combinations, as T7 RNA polymerase is not expressed until IPTG (isopropyl thio-β-D-galactoside) is added as an expression inductor, the target protein downstream of the T7 promoter is not expressed, therefore the stress on E. coli due to overexpression is reduced. Further, since Plasmid pLysS expresses T7 lysozyme and inactivates T7 RNA polymerase, a two-step inhibition can be expected. In this invention, it is preferred to select an expression vector from expression vectors including T7lac promoter, and it is particularly preferred to use pET30a.

After expression induction, the stress on E. coli is reduced using a culture medium selected from composite culture media by optimizing growth conditions, such as culture temperature, IPTG addition concentration and pH. In this invention, by deliberately removing the growth conditions which are optimal for the growth of E. coli, the expression of the target protein can be smoothly promoted, long-term growth is possible and the yield of target protein can be increased. Therefore, in this invention, it is preferred to set the culture temperature 2-7° C. lower than the optimum growth temperature of E. coli.

It is particularly preferred that the culture medium used in this invention is TB culture medium. The IPTG addition concentration is preferably 0.2-11.0 mM. The pH is preferably 6.7-7.0.

### EXAMPLES

Hereafter, this invention will be described by means of specific examples, although the invention is not to be construed as being limited in any way thereby. In addition, unless otherwise stated, “%” means “weight %” and ratio means weight ratio. The meanings of the symbols in the text are as given below.

### Example 1

<Construction of SLP Gene>

The four oligonucleotides shown in the SEQ ID NOS: 12-15 of Asahi TechnoGlass, were designed.

The synthesized film-like oligonucleotides were dissolved so that their concentration was 1 μg/μl using TrisEDTA (10 mM Tris-HCl (pH 8.0), 1 mM EDTA (pH 8.0): hereafter, TE). Two double-stranded DNAs which code for the aminoacids expressed in the SEQ ID NOS: 16 and 17 were constructed by equimolar mixing of complementary strands, heat-treating at 99° C. for 30 seconds, cooling at 37° C. for 1 hour, and settling for 30 minutes. After mixing equivalent amounts of the double-stranded DNAs, ligation was performed at 16° C. for 1 hour using Takara Ligation Kit ver2 solution I (product of TAKARA SHUZO CO., LTD.) so as to prepare double-stranded DNA which codes for the SLP monomer (SLP monomer sequence; ThrSer[GlyGlyAlaGlySerGlyTyrGlyGlyGlyTyrGlyHisGly TyrGlySerAspGlyGly(GlyAlaGlyAlaGlySer)₃AlaSer]ₙ (n=2, 4, 6) (see SEQ ID NO: 18 (for n=2)).

The cloning vector pUC118 (product of TAKARA SHUZO CO., LTD.) was digested at 37° C. for 1 hour 30 minutes using the restriction enzyme BamHI, CIAP (Calf Intestine Alkaline Phosphatase) (product of TAKARA SHUZO CO., LTD.) was added, and treatment was performed at 37° C. for 30 minutes (hereafter, “alkaline phosphatase treatment”). The reaction solution was extracted and purified with a mixture of phenol:chloroform:isoamyl alcohol in a ratio of 25:24:1 (weight ratio). Ethanol was added to the purified reaction solution, and the resulting precipitate was used as the vector sample.

The monomer DNA of SLP and a pUC118 vector sample were mixed in a ratio of 10:1 (weight ratio), and ligation was performed at 16° C. for 1 hour using Takara Ligation Kit ver2 solution I. After completion of the reaction, transformation was performed using competent cell DH5α. The presence or absence of an insert gene was verified by color selection using X-gal (5-bromo-4-chloro-3-indolyl-β-D-galactopyranoside), DNA sequencing was performed on material containing the insert gene, and plasmid PUC-SLP (1) containing the monomer DNA of SLP was obtained by verifying the sequence.

<Construction of PUC-Link>

The cloning vector pUC118 used for this study does not include the regions digested with the restriction enzymes Nhe I and Spe I. Therefore, an adapter was designed for the purpose of adding the recognition regions of the restriction enzymes Nhe I and Spe I to pUC118 (SEQ ID NO: 19), and a pUC118-Link (plasmid containing the designed adapter) was constructed. Codons which code methionine residues other than the recognition regions of the restriction enzymes Nhe I and Spe I were arranged on both sides in the adapter. Methionine residues were thereby added on both sides of the insert gene of the expressed protein obtained, and a sample which did not include a sequence of plasmid origin was obtained by specifically cleaving the methionine residues using cyanogen bromide.

The synthesized film-like oligonucleotides were dissolved so that their concentration was 1 μg/μl using TE (10 mM Tris-HCl (pH 8.0), 1 mM EDTA (pH 8.0)). Double-stranded DNA which codes for the aminoacids expressed in the SEQ ID NOS: 16 and 17 was constructed by equimolar mixing of complementary strands, heat-treating at 99° C. for 30 seconds, cooling at 37° C. over 1 hour, and settling for 30 minutes. After mixing equivalent amounts of the respective double stranded DNAs, the cloning vector pUC118 (product of TAKARA SHUZO CO., LTD.) was digested at 37° C. for 1 hour 30 minutes using the restriction enzyme Xba I, CIAP was added, and treatment was performed at 37° C. for 30 minutes. The reaction solution was extracted and purified using phenol:chloroform:isoamyl alcohol in a ratio of 25:24:1 (weight ratio). Ethanol was added to the purified reaction liquid, and the precipitate produced was dissolved in sterilized water for use as the vector sample.

The pUC118 vector sample was mixed with Adapter DNA in a ratio of 10:1 (weight ratio), and ligation was performed at 16° C. for 1 hour using Takara Ligation Kit ver2 Solution I. After the reaction was complete, transformation was performed using competent cell DH5α. Plasmid pUC-Link containing the adapter was obtained by verifying the presence or absence of the insert gene, performing DNA sequencing on the material containing the insert gene, and verifying the sequence by color selection using X-gal.

<Construction of SLP(n)>

The restriction enzyme recognition regions of Spe I and Nhe I are included at both ends of the SLP monomer. The projecting ends of the fragments digested with restriction enzymes Spe I and Nhe I are all complementary and can be mutually combined. The newly combined sequences are all different from the restriction enzyme recognition regions of Spe I and Nhe I, and are not digested by Spe I and Nhe I. Using this property, plasmid pUC-Link SLP (n) containing DNA which polymerizes SLP monomer in one sense and codes SLP n times, was constructed.

Competent cell DH5α was transformed by PUC-SLP (1), and cultured in 2xYT culture medium at 37° C. for 18 hours. The plasmid was extracted from the liquid culture medium by the alkali-SDS method, and dissolved in TE. The sample was simultaneously digested by Nhe I and Spe I (both products of TAKARA SHUZO CO., LTD.) at 37° C. for 1 hour 30 minutes, and SLP (1) was isolated from the plasmid. After concentrating the reaction liquid to 5 μl using MicroCon (product of Millipore CO., LTD.), electrophoresis was performed using a 1.5% agarose gel, and the band of insert DNA was cut out. To extract DNA from the gel, the extract was again concentrated to 5 μl using Ultrafree DA (product of Millipore CO., LTD.), and this was used as the insert genetic material.

After digesting pUC-Link by Nhe I, CIAP was added and alkaline phosphatase treatment was carried out. The reaction solution was extracted and purified using a mixture of phenol:chloroform:isoamyl alcohol (weight ratio 25:24:1). Ethanol was added to the purified reaction solution, the precipitate produced was dissolved in sterilized water, and this was used as the vector sample.

The DNA concentrations in the insert genetic material and vector sample were verified by electrophoresis using 1.5% agarose gel, the insert genetic material and vector sample were mixed in a ratio of 10:1, an equivalent amount of Takara Ligation Kit ver2 Solution I to the reaction mixture was added, and ligation was performed at 16° C. for 1 hour.

After the reaction was complete, transformation of competent cell DH5α was performed. It was then inoculated on a LB plate with added ampicillin, and screened. The colony produced was picked up, inoculated on 2xYT culture medium and grown at 37° C. for 18 hours. From the culture medium, the plasmid was extracted by the alkali-SDS miniprep method, dissolved in TE and used as a sample. After digesting the sample simultaneously using Nhe I and Spe I, the presence or absence and size of the insert gene were verified by the electrophoresis method. Then, DNA sequencing was performed and plasmid pUC-LinkSLP (1) was obtained by verifying the sequence.

After digesting pUC-Link SLP (1) by Nhe I, CIAP was added and alkaline phosphatase solution treatment was carried out. The reaction solution was extracted and purified using a mixture of phenol:chloroform:isoamyl alcohol (weight ratio 25:24:1), ethanol was added to the purified reaction solution, the precipitate obtained was dissolved in sterilized water, and this was used as the vector sample.

The DNA concentration in the insert genetic material and vector sample was verified by electrophoresis using 1.5% agarose gel, the insert genetic material and vector sample were mixed in a ratio of 10:1, an equivalent amount of Takara Ligation Kit ver2 Solution I to the reaction mixture was added, and ligation was performed at 16° C. for 1 hour.

After the reaction was complete, transformation of competent cell DH5α was performed. It was then inoculated on a LB plate with added ampicillin, and screened. The colony produced was picked up, inoculated on 2xYT culture medium and grown at 37° C. for 18 hours. From the culture medium, the plasmid was extracted by the alkali-SDS miniprep method, dissolved in TE and used as a sample. After digesting the sample simultaneously using Nhe I and Spe I, the presence or absence and size of the insert gene were verified by electrophoresis. Then, DNA sequencing was performed and plasmid pUC-Link SLP (2) (dimer) was obtained by verifying the sequence.

SLP (2) was inserted in pUC-Link SLP (2) to give pUC-Link SLP (4) (tetramer), then SLP (2) was inserted in pUC-Link SLP (4) to give pUC-Link SLP (6) (hexamer).

<Construction of Expression Vector pET-SLP (N)>

pUC-Link SLP (2, 4, 6) obtained as mentioned above was digested using the restriction enzymes BamHI and Hind III (both products of TAKARA SHUZO CO., LTD.). After concentrating the reaction liquid to 5 μl using MicroCon (product of Millipore CO., LTD.), electrophoresis was performed using a 1.5% agarose gel, and the band of insert DNA was cut out. To extract DNA from the gel, the extract was concentrated to 5 μl using Ultrafree DA and then MicroCon, and this was used as the insert genetic material.

After digesting the expression vector pET30a (product of Novagen CO., LTD.) by Nhe I, CIAP was added and alkaline phosphatase treatment was carried out. The reaction solution was extracted and purified using a mixture of phenol:chloroform:isoamyl alcohol (weight ratio 25:24:1). Ethanol was added to the purified reaction solution, the precipitate produced was dissolved in sterilized water, and this was used as the vector sample.

The DNA concentrations in the insert genetic material and vector sample were verified by electrophoresis using 1.5% agarose gel, the insert genetic material and vector sample were mixed in a ratio of 10:1, an equivalent amount of Takara Ligation Kit ver2 Solution I to the reaction mixture was added, and ligation was performed at 16° C. for 1 hour.

After the reaction was complete, transformation of competent cell DH5α was performed. It was then inoculated on a LB plate with added kanamycin, and screened. The colony produced was picked up, inoculated on 2xYT culture medium and grown. From the culture medium, the vector was extracted by the alkali-SDS method, dissolved in TE and used as a sample. After digesting the sample simultaneously using Nhe I and Spe I, the presence or absence and size of the insert gene were verified by the electrophoresis method. Then, DNA sequencing was performed and the expression vector pET-SLP (2, 4, 6) was obtained by verifying the sequence.

<Expression of pET-SLP (2, 4, 6)>

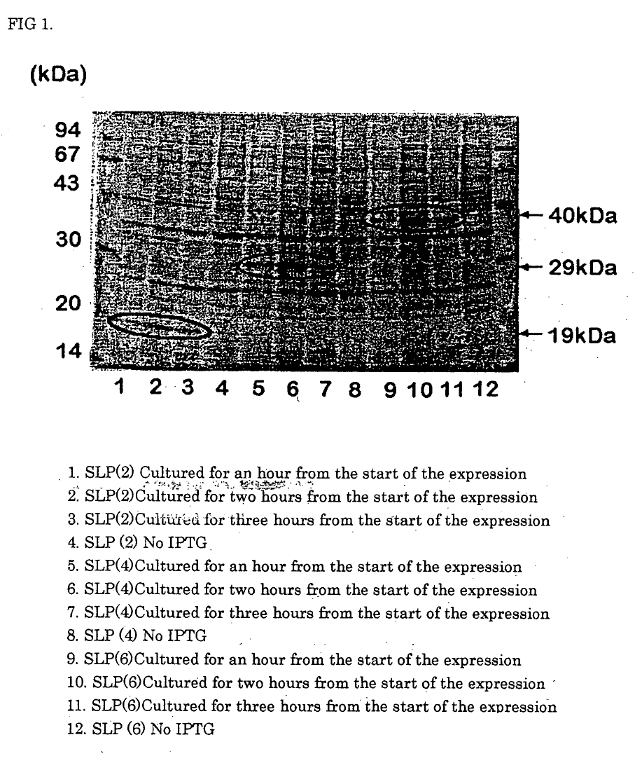

Host E. coli BL21(DE3) pLysS (product of Novagen CO., LTD.) containing each of the aforesaid plasmids pET-SLP (2, 4, 6) obtained as mentioned above was grown at 37° C. for 16 hours in 1 ml of 2xTY (25 μg/ml kanamycin, 25 μg/ml chloramphenicol) liquid culture medium. Next, 100 μl of the culture medium was added to a L-shaped tube containing 5 ml of 2xTY (25 μg/ml kanamycin, 25 μg/ml chloramphenicol), and cultured at 37° C. for 1 hour (OD₆₀₀=0.5−0.7 (Shimadzu UV 160)). In this case, to induce the expression of SLP, IPTG (final concentration 1 mM) was added, 100 μl of culture medium was sampled in an Eppendorf tube every other hour, and cultured for 3 hours. After performing centrifugal separation (14500 rpm, 5 minutes, 4° C.) of the sampled culture medium, the supernatant liquid was discarded, pellets were dissolved in 2× sample buffer (buffer solution for sample dissolution), and heat-treated at 100° C. for 5 minutes to give a SDS-PAGE sample. As shown in FIG. 1, unique bands depending on IPTG were respectively observed at 19 kDa for SLP 2, 29 kDa for SLP 4 and 40 kDa for SLP 6. From this, it was confirmed that SLP genes were induced by IPTG addition, and mass-expressed strains could be obtained.

Next, host E. coli BL21(DE3) pLysS respectively containing the plasmids PET-SLP (2, 4, 6) was grown at 37° C. for 16 hours in 2.5 ml 2xTY (25 μg/ml kanamycin, 25 μg/ml chloramphenicol) liquid culture medium. Next, this culture medium was added to a 500 ml flask containing 250 ml 2xTY(s) (25 μg/ml kanamycin, 25μ/ml chloramphenicol), and the culture medium was cultured at 37° C. for 1 hour (OD₆₀₀=0.5−0.7 (Shimadzu UV-160)). In this case, to induce proteinic expression, IPTG (final concentration 1 mM) was added, and a bacteria was obtained by growing for a further 2 hours and collecting (5000 rpm, 10 minutes, 4° C.). The obtained bacteria was stored at −30° C.

This bacteria stored at −30° C. was slowly thawed on ice, suspended in Lysis buffer (buffer solution for protein dissolution) (50 mM Tris-HCl, 300 mM NaCl, 10 mM imidazole), and ultrasonic crushing (Output 3.5, Duty 60% (TOMY UD201)) was performed on ice 4 times, 1 minute at a time, with a cooling time of 1 minute. Centrifugal separation (10,000 rpm, 10 minutes, 4° C.) of the bacterial crushed solution was performed, and the supernatant liquid was collected.

The obtained supernatant liquid was used as an addition sample, and purified by affinity chromatography (flow velocity 15-20 ml/hour) using a column filled with nickel-NTA agarose beads equilibrated beforehand using the same buffer solution. The eluates were fractionated, and the fractions containing the target protein were verified and recovered by SDS-PAGE.

After dialyzing the obtained fractions while exchanging with distilled water as the outside liquid as required for 24 to 48 hours, a white powder was obtained by freeze-drying. The yields of each protein of molecular weight 19 kDa, 29 kDa, 40 kDa are shown in Table 1.

TABLE 1

| Sample | Yield (mg) | Yield (%) |
|---|---|---|
| SLP 6 | 21 | 52.5 |
| SLP 4 | 24 | 60.0 |
| SLP 2 | 15 | 50.0 |

<Identification of SLP>

For each protein, the aminoacid sequences of the N-terminal residues were determined by the N-terminal aminoacid sequences. The protein was dissociated from the surrounding proteins by the electrophoresis method using polyacrylamide gel, and transferred to a PVDF (polyvinylidene fluoride) film using SART BLOT 2-S (product of SARTORIUS CO., LTD.). After transfer, and staining with staining solution for 5 minutes, the target protein was cut out using scissors which had been bleached and rinsed with methanol. Using this sample, the N-terminal aminoacid sequence was determined using an ABI 473 gaseous-phase Edman sequencer. The result coincided with the expected aminoacid sequence, and it was confirmed that the expressed protein is a protein of plasmid origin.

<Cleavage of Tag Sequence by Cyanogen Bromide>

In the designed SLP 2, 4, 6, methionine residues are arranged on both sides of the insert gene by an adapter. Since SLP 2, 4, 6 do not themselves contain methionine residues, a protein which does not include an aminoacid sequence of plasmid origin, such as a tag, can be obtained by chemically cleaving amethionine residue. Proteinic methionine residues were cleaved, and the aminoacid sequences of the N terminal residues were determined by the N-terminal aminoacid sequence.

10 mg of SLP6 was taken in an Eppendorf tube, and dissolved in 90% formic acid. After confirming that it had dissolved completely, it was diluted with milli Q water (ultrapure water) until the final concentration of formic acid was 70%. After adding 10 mg cyanogen bromide to the sample solution and dissolving, it was shaded completely with aluminum foil and left at room temperature for 12 to 48 hours. After adding 10 times the amount of milli Q water to the reaction solution and stopping the reaction, it was dialyzed with distilled water as the outside liquid, and a white powder was then obtained by freeze-drying. When the obtained white powder was dissolved in 2× sample buffer and the molecular weight was compared by SDS-PAGE, a reduction in molecular weight was found as compared with the sample before cyanogen bromide treatment.

The SLP 6 purified in this way was isolated from impure proteins by the electrophoresis method using polyacrylamide gel, and transferred to a PVDF film using SART BLOT 2-S (SARTORIUS). After transfer, and staining with staining solution for 5 minutes, the target protein was cut out using scissors which had been bleached and rinsed with methanol. Using this sample, the N-terminal aminoacid sequence was determined using an ABI 473 gaseous-phase Edman sequencer. The determined N-terminal aminoacid sequence coincided with the expected aminoacid sequence, and was confirmed to be the target protein SLP 6.

### Example 2

<Construction of SLPA Gene>

The four oligonucleotides shown in the SEQ ID NOS: 20-23 synthesized by Asahi TechnoGlass CO., LTD., were designed.

The synthesized film-like oligonucleotides were dissolved so that their concentration was 1 μg/μl using TE (10 mM Tris-HCl (pH 8.0), 1 mM EDTA (pH 8.0): hereafter, TE). Two double-stranded DNAs which code for the aminoacids expressed in the SEQ ID NOS: 24 and 25 were constructed by equimolar mixing of complementary strands, heat-treating at 99° C. for 30 seconds, cooling at 37° C. for 1 hour, and settling for 30 minutes.

The cloning vector pUC118 (product of TAKARA SHUZO CO., LTD.) was digested at 37° C. for 1 hour 30 minutes using the restriction enzyme BamHI, CIAP (Calf Intestine Alkaline Phosphatase) (product of TAKARA SHUZO CO., LTD.) was added, and treatment was performed at 37° C. for 30 minutes (hereafter, “alkaline phosphatase treatment”). The reaction solution was extracted and purified with a mixture of phenol:chloroform:isoamyl alcohol in a ratio of 25:24:1 (weight ratio). Ethanol was added to the purified reaction solution, and the resulting precipitate was used as the vector sample.

The double-stranded DNA and pUC118 vector sample were mixed in a ratio of 10:1 (weight ratio), and ligation was performed at 16° C. for 1 hour using Takara Ligation Kit ver2 solution I to prepare double-stranded DNA which codes for the aminoacid sequences in SEQ ID NOS: 24 and 25. After completion of the reaction, transformation was performed using competent cell DH5 α. The presence or absence of an insert gene was verified by color selection using X-gal, DNA sequencing was performed on material containing the insert gene, and plasmid pUC-ALA containing the DNA sequence coding the polyalanine (SEQ ID NO: 24) with plasmid pUC-GX containing the DNA sequence coding the alternating copolymer of glycine (X=Ala, Tyr, Val) (SEQ ID NO: 25), were obtained by verifying the sequences.

pUC-ALA was transformed using competent cell DH5α, and cultured in 2xYT culture medium at 37° C. for 18 hours. The plasmid was extracted from the culture medium by the alkali-SDS method, and dissolved in TE. The sample was simultaneously digested by Nhe I and Spe I at 37° C. for 1 hour 30 minutes, and ALA was isolated from the plasmid. After concentrating the reaction liquid to 5 μl using MicroCon (product of Millipore CO., LTD.), electrophoresis was performed using a 1.5% agarose gel, and the band of insert DNA was cut out. To extract DNA from the gel, the extract was concentrated to 5 μl using Ultrafree DA and then MicroCon, and this was used as the insert genetic material.

After digesting pUC-GX by Spe I, CIAP was added and alkaline phosphatase treatment was carried out. The reaction solution was extracted and purified using a mixture of phenol:chloroform:isoamyl alcohol (weight ratio 25:24:1). Ethanol was added to the purified reaction solution, the precipitate produced was dissolved in sterilized water, and this was used as the vector sample.

The DNA concentrations in the insert genetic material and vector sample were verified by electrophoresis using 1.5% agarose gel, the insert genetic material and vector sample were mixed in a ratio of 10:1, an equivalent amount of Takara Ligation Kit ver2 Solution I to the reaction mixture was added, and ligation was performed at 16° C. for 1 hour.

After the reaction was complete, transformation of competent cell DH5α was performed. It was then inoculated on a LB plate with added ampicillin, and screened. The colony produced was picked up, inoculated on 2xYT culture medium and grown at 37° C. for 18 hours. From the culture medium, the plasmid was extracted by the alkali-SDS miniprep method, dissolved in TE and used as a sample. After digesting the sample simultaneously using Nhe I and Spe I, the presence or absence and size of the insert gene were verified by the electrophoresis method. Then, DNA sequencing was performed and plasmid pUC-SLPA (1) was obtained by verifying the sequence (aminoacid sequence of SLPA: AlaSer [(Ala) ₁₈ThrSerGlyValGlyAlaGlyTyrGlyAlaGlyAlaGlyTyrGlyV alGlyAlaGlyTyrGlyAlaGlyValGlyTyrGlyAlaGlyAlaGlyTyrThrSer]ₙ, SEQ ID NO: 26 (for n=4)).

<Construction of SLPA(n)>

Competent cell DH5α was transformed by pUC-SLPA (1), and cultured in 2xYT culture medium at 37° C. for 18 hours. The plasmid was extracted from the culture medium by the alkali-SDS method, and dissolved in TE. The sample was simultaneously digested by Nhe I and Spe I at 37° C. for 1 hour 30 minutes, and SLPA (1) was isolated from the plasmid. After concentrating the reaction liquid to 5 μl using MicroCon, electrophoresis was performed using 1.5% agarose gel, and the band of insert DNA was cut out. To extract DNA from the gel, the extract was concentrated to 5 μl using UltrafreeDA and then MicroCon, and this was used as the insert genetic material.

After digesting pUC-SLPA (1) by Nhe I, CIAP was added and alkaline phosphatase treatment was carried out. The reaction solution was extracted and purified using a mixture of phenol:chloroform:isoamyl alcohol (weight ratio 25:24:1). Ethanol was added to the purified reaction solution, the precipitate produced was dissolved in sterilized water, and this was used as the vector sample.

The DNA concentrations in the insert genetic material and vector sample were verified by electrophoresis using 1.5% agarose gel, the insert genetic material and vector sample were mixed in a ratio of 10:1, an equivalent amount of Takara Ligation Kit ver2 Solution I to the reaction mixture was added, and ligation was performed at 16° C. for 1 hour.

After the reaction was complete, transformation of competent cell DH5α was performed. It was then inoculated on a LB plate with added ampicillin, and screened. The colony produced was picked up, inoculated on 2xYT culture medium and grown at 37° C. for 18 hours. From the culture medium, the plasmid was extracted by the alkali-SDS miniprep method, dissolved in TE and used as a sample. After digesting the sample simultaneously using Nhe I and Spe I, the presence or absence and size of the insert gene were verified by the electrophoresis method. Then, DNA sequencing was performed and plasmid pUC-SLPA (2) was obtained by verifying the sequence.

SLP (2) was inserted in pUC-SLPA (2) to give pUC-SLPA (4) (tetramer).

<Construction of Expression Vector pET-SLPA (4)>

pUC-SLAP (4) was digested with BamHI. After concentrating the reaction liquid to 5 μl using MicroCon, electrophoresis was performed using 1.5% agarose gel, and the band of insert DNA was cutout. To extract DNA from the gel, the extract was concentrated to 5 μl using UltrafreeDA and then MicroCon, and this was used as the insert genetic material.

After digesting pET30a by BamHI, CIAP was added and alkaline phosphatase treatment was carried out. The reaction solution was extracted and purified using a mixture of phenol:chloroform:isoamyl alcohol (weight ratio 25:24:1). Ethanol was added to the purified reaction solution, the precipitate produced was dissolved in sterilized water, and this was used as the vector sample.

The DNA concentrations in the insert genetic material and vector sample were verified by electrophoresis using 1.5% agarose gel, the insert genetic material and vector sample were mixed in a ratio of 10:1, an equivalent amount of Takara Ligation Kit ver2 Solution I to the reaction mixture was added, and ligation was performed at 16° C. for 1 hour.

After the reaction was complete, transformation of competent cell DH5α was performed. It was then inoculated on a LB plate with added kanamycin, and screened. The colony produced was picked up, inoculated on 2xYT culture medium and grown at 37° C. for 18 hours. From the culture medium, the plasmid was extracted by the alkali-SDS miniprep method, dissolved in TE and used as a sample. After digesting the sample simultaneously using Nhe I and Spe I, the presence or absence and size of the insert gene were verified by the electrophoresis method. Then, DNA sequencing was performed and the expression vector PET-SLPA (4) was obtained by verifying the sequence.

<Expression of pET-SLPA (4)>

Host E. coli BL21 (DE3) pLysS containing each of the aforesaid plasmids pET-SLPA (4) was grown at 37° C. for 16 hours in 1.5 ml of 2xTY (25 μg/ml kanamycin, 25 μg/ml chloramphenicol) liquid culture medium. Next, 100 μl of the culture medium was added to a test tube containing 5 ml of 2xTY (25 μg/ml kanamycin, 25 μg/ml chloramphenicol), and cultured at 37° C. for 1 hour (OD600=0.5−0.7 (Shimadzu UV 160)). In this case, to induce the expression of SLP, IPTG (final concentration 1 mM) was added, 100 μl of culture medium was sampled in an Eppendorf tube every other hour, and cultured for 4 hours. After performing centrifugal separation (14500 rpm, 5 minutes, 4° C.) of the sample culture medium, the supernatant liquid was discarded, pellets were dissolved in 2× sample buffer (buffer solution for sample dissolution), and heat-treated at 100° C. for 5 minutes to give a SDS-PAGE sample.

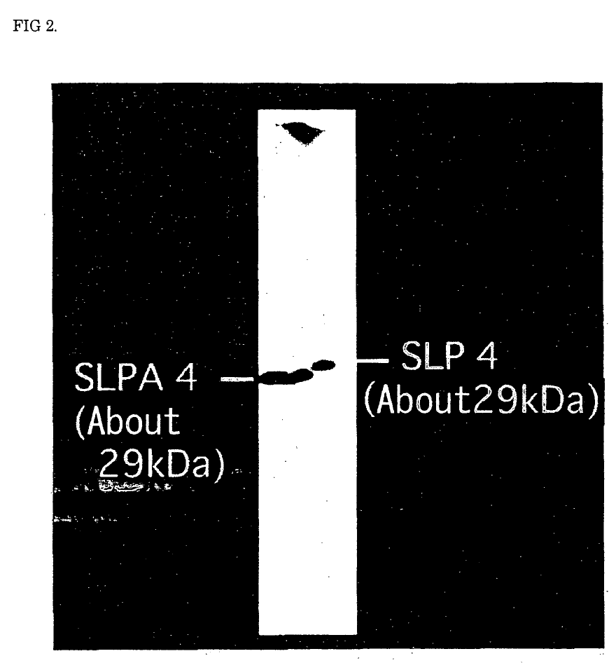

After obtaining the SDS-PAGE sample, SPLA 4 was detected by Western Blot using His-Tag (FIG. 2).

As can be seen from the figure, with SLPA 4, a band was observed at 29 kDa. From this, it was confirmed that SLP genes were induced by IPTG addition, and mass-expressed strains could be obtained.

Host E. coli BL21 (DE3) pLysS containing the plasmid pET-SLPA (4) was grown at 37° C. for 16 hours in 1.5 ml 2xTY (25 μg/ml kanamycin, 25 μg/ml chloramphenicol) liquid culture medium. Next, this culture medium was added to a test tube containing 12 ml 2xTY (s) (25 μg/ml kanamycin, 25μ/ml chloramphenicol), and the culture medium was cultured at 37° C. for 16 hours. Next, it was added to a 21 fermenter containing 1.2 l 2xYT (25 μg/ml kanamycin, 25 μg/ml chloramphenicol) liquid culture medium), and cultured at 37° C. until OD600=0.5−0.7 (Shimadzu UV 160). In this case, IPTG (final concentration 1 mM) was added to induce expression of the protein, and a bacteria was obtained by growing for a further 4 hours and collecting (8500 rpm, 30 minutes, 4° C.). The obtained bacteria was stored at −20° C.

The aforesaid bacteria stored at −20° C. was slowly thawed on ice, suspended in Lysis buffer (50 mM Tris-HCl, 300 mM NaCl, 10 mM imidazole), and ultrasonic crushing (Output 3.5, Duty 60% (TOMY UD201)) was performed on ice 20 times, 2 minutes at a time, with a cooling time of 1 minute. Centrifugal separation (10,000 rpm, 40 minutes, 4° C.) of the crushed bacterial solution was performed, and a precipitate was recovered.

Buffer B (100 mM NaH₂PO₄, 10 mM Tris-Cl, 8M urea, pH 8.0) was added to the obtained precipitate, and ultrasonic crushing was performed. The crushed bacterial solution obtained here was centrifuged (10,000 rpm, 40 minutes, 4° C.), and the supernatant liquid was recovered.

The obtained supernatant liquid was used as an addition sample, and purified by affinity chromatography (flow velocity 15-20 ml/hour) using a column filled with Ni-NTA agarose beads equilibrated beforehand using the same buffer solution. The eluates were fractionated, and the fractions containing the target protein were verified and recovered by SDS-PAGE.

After dialyzing the obtained fractions while exchanging with distilled water as the outside liquid as required for 24 to 48 hours, a white powder was obtained by freeze-drying. The yield was 34.2 mg/L.

### Example 3

<Construction of SELP Gene>

The four oligonucleotides shown in the SEQ ID NOS: 27-30 synthesized by Asahi TechnoGlass CO., LTD., were designed.

The synthesized film-like oligonucleotides were dissolved so that their concentration was 1 μg/μl using TE (10 mM Tris-HCl (pH 8.0), 1 mM EDTA (pH 8.0); hereafter, TE). Double-stranded DNAs which code for the aminoacids expressed in the SEQ ID NOS: 31 and 32 were constructed by equimolar mixing of complementary strands, heat-treating at 99° C. for 30 seconds, cooling at 37° C. for 1 hour, and settling for 30 minutes. After mixing equivalent amounts of the respective double-stranded DNAs, ligation was performed at 16° C. for 1 hour using Takara Ligation Kit ver2 Solution I to prepare a typical double-stranded DNA coding for the SELP monomer (aminoacid sequence of SELP: ThrSer[(Gly Val Pro Gly Val)₂ Gly Gly(Gly Ala Gly Ala Gly Ser)₃ Ala Ser]ₙ, SEQ ID NO: 33 (for n=8)).

The cloning vector pUC118 was digested at 37° C. for 1 hour 30 minutes using the restriction enzyme BamHI, CIAP was added, and treatment was performed at 37° C. for 30 minutes. The reaction solution was extracted and purified with a mixture of phenol:chloroform:isoamyl alcohol in a ratio of 25:24:1 (weight ratio). Ethanol was added to the purified reaction solution, the precipitate produced was dissolved in sterilized water, and used as the vector sample.

The SELP monomer DNA and pUC118 vector sample were mixed in a ratio of 10:1 (weight ratio), and ligation was performed at 16° C. for 1 hour using Takara Ligation Kit ver2 solution. After completion of the reaction, transformation was performed using competent cell DH5α. The presence or absence of an insert gene was verified by color selection using X-gal, DNA sequencing was performed on material containing the insert gene, and plasmid pUC-SELP (1) containing the SELP monomer DNA was obtained by verifying the sequence.

<Construction of SELP(n)>

Complement cell DH5α was transformed using PUC-SELP (1), and cultured in 2xYT culture medium at 37° C. for 18 hours. The plasmid was extracted from the culture medium by the alkali-SDS method, and dissolved in TE. The sample was simultaneously digested by Nhe I and Spe I at 37° C. for 1 hour 30 minutes, and SLP (1) was isolated from the plasmid. After concentrating the reaction liquid to 5 μl using MicroCon, electrophoresis was performed using 1.5% agarose gel, and the band of insert DNA was cut out. To extract DNA from the gel, the extract was concentrated to 5 μl using UltrafreeDA and then MicroCon, and this was used as the insert genetic material.

After digesting pUC-Link by Nhe I, CIAP was added and alkaline phosphatase treatment was carried out. The reaction solution was extracted and purified using a mixture of phenol:chloroform:isoamyl alcohol (weight ratio 25:24:1). Ethanol was added to the purified reaction solution, the precipitate produced was dissolved in sterilized water, and this was used as the vector sample.

The DNA concentrations in the insert genetic material and vector sample were verified by electrophoresis using 1.5% agarose gel, the insert genetic material and vector sample were mixed in a ratio of 10:1, an equivalent amount of Takara Ligation Kit ver2 Solution I to the reaction mixture was added, and ligation was performed at 16° C. for 1 hour.

After the reaction was complete, transformation of competent cell DH5α was performed. It was then inoculated on a LB plate with added ampicillin, and screened. The colony produced was picked up, inoculated on 2xYT culture medium and grown at 37° C. for 18 hours. The plasmid was extracted from the culture medium by the alkali-SDS miniprep method, dissolved in TE and used as a sample. After digesting the sample simultaneously using Nhe I and Spe I, the presence or absence and size of the insert gene were verified by the electrophoresis method. Then, DNA sequencing was performed and plasmid pUC-Link SELP (1) was obtained by verifying the sequence.

Competent cell DH5α was transformed using pUC-SELP (1), and cultured in 2xYT culture medium at 37° C. for 18 hours. The plasmid was extracted from the culture medium by the alkali-SDS method, and dissolved in TE. The sample was simultaneously digested by Nhe I and Spe I at 37° C. for 1 hour 30 minutes, and SELP (1) was isolated from the plasmid. After concentrating the reaction liquid to 5 μl using MicroCon, electrophoresis was performed using 1.5% agarose gel, and the band of insert DNA was cutout. To extract DNA from the gel, the extract was concentrated to 5 μl using UltrafreeDA and then MicroCon, and this was used as the insert genetic material.

After digesting pUC-Link SELP (1) by Nhe I, CIAP was added and alkaline phosphatase treatment was carried out. The reaction solution was extracted and purified using a mixture of phenol:chloroform:isoamyl alcohol (weight ratio 25:24:1). Ethanol was added to the purified reaction solution, the precipitate produced was dissolved in sterilized water, and this was used as the vector sample.

The DNA concentrations in the insert genetic material and vector sample were verified by electrophoresis using 1.5% agarose gel, the insert genetic material and vector sample were mixed in a ratio of 10:1, an equivalent amount of Takara Ligation Kit ver2 Solution I to the reaction mixture was added, and ligation was performed at 16° C. for 1 hour.

After the reaction was complete, transformation of competent cell DH5α was performed. It was then inoculated on a LB plate with added ampicillin, and screened. The colony produced was picked up, inoculated on 2xYT culture medium and grown at 37° C. for 18 hours. The plasmid was extracted from the culture medium by the alkali-SDS miniprep method, dissolved in TE and used as a sample. After digesting the sample simultaneously using Nhe I and Spe I, the presence or absence and size of the insert gene were verified by the electrophoresis method. Then, DNA sequencing was performed and plasmid pUC-Link SELP (2) was obtained by verifying the sequence.

SELP (2) was inserted in pUC-Link SELP (2) to construct pUC-Link SELP (4), and SELP (4) was inserted in pUC-Link SELP (4) to construct pUC-Link SELP (8).

<Construction of Expression Vector pET-SELP(n)>

PUC-SELP (8) was digested using BamHI and Hind III. After concentrating the reaction liquid to 5 μl using MicroCon, electrophoresis was performed using a 1.5% agarose gel, and the band of insert DNA was cut out. To extract DNA from the gel, the extract was concentrated to 5 μl using UltrafreeDA and then MicroCon, and this was used as the insert genetic material.

After digesting the expression vector pET30a by BamHI and Hind III, CIAP was added and alkaline phosphatase treatment was carried out. The reaction solution was extracted and purified using a mixture of phenol:chloroform:isoamyl alcohol (weight ratio 25:24:1). Ethanol was added to the purified reaction solution, the precipitate produced was dissolved in sterilized water, and this was used as the vector sample.

The DNA concentrations in the insert genetic material and vector sample were verified by electrophoresis using 1.5% agarose gel, the insert genetic material and vector sample were mixed in a ratio of 10:1, an equivalent amount of Takara Ligation Kit ver2 Solution I to the reaction mixture was added, and ligation was performed at 16° C. for 1 hour.

Transformation of competent cell DH5α was performed using ligation reaction solution. It was then inoculated on a LB plate with added kanamycin, and screened. The colony produced was picked up, inoculated on 2xYT culture medium and grown. From the culture medium, the vector was extracted by the alkali-SDS method, dissolved in TE and used as a sample. After digesting the sample simultaneously using Nhe I and Spe I, the presence or absence and size of the insert gene were verified by the electrophoresis method. Then, DNA sequencing was performed and the expression vector pET-SELP 8 was constructed by verifying the sequence.

<Expression of pET-SELP (8)>

Host E. coli BL21(DE3) pLysS containing the respective plasmids pET-SELP (8) obtained as mentioned above was grown at 37° C. for 16 hours in 1.5 ml of 2xTY (25 μg/ml kanamycin, 25 μg/ml chloramphenicol) liquid culture medium. Next, 100 μl of the culture medium was added to a test-tube containing 5 ml of 2xTY (25 μg/ml kanamycin, 25 μg/ml chloramphenicol), and cultured at 37° C. until OD₆₀₀=0.5−0.7. In this case, to induce the expression of SELP 8, IPTG (final concentration 1 mM) was added, 100 μl of culture medium was sampled in an Eppendorf tube every other hour, and cultured for 4 hours. After performing centrifugal separation (14500 rpm, 5 minutes, 4° C.) of the sampled culture medium, the supernatant liquid was discarded, pellets were dissolved in 2× sample buffer, and heat-treated at 100° C. for 5 minutes to give a SDS-PAGE sample.

After obtaining the SDS-PAGE sample, SELP 8 was detected by performing Western Blot using His-Tag antibody.

As shown in the figure, with SELP 8, a band was observed at 35 kDa. From this, it was confirmed that SLP genes were induced by IPTG addition, and mass-expressed strains could be obtained.

Host E. coli BL21(DE3) pLysS respectively containing the plasmids pET-SELP (8) was grown at 37° C. for 16 hours in 1.5 ml 2xTY (25 μg/ml kanamycin, 25 μg/ml chloramphenicol) liquid culture medium. Next, this culture medium was cultured at 37° C. for 16 hours in 12 ml of 2 xYT (25 μg/ml kanamycin, 25u/ml chloramphenicol) liquid culture medium. Next, it was added to a 21 fermenter containing 1.2 l 2xYT (25 μg/ml kanamycin, 25 μg/ml chloramphenicol) liquid culture medium, and cultured at 37° C. until OD₆₀₀=0.5−0.7. In this case, IPTG (final concentration 1 mM) was added to induce expression of the protein, and a bacteria was obtained by lowering the temperature to 30° C., culturing for a further 4 hours and collecting (8500 rpm, 30 minutes, 4° C.). The obtained bacteria was stored at −20° C.

The aforesaid bacteria stored at −20° C. was slowly thawed on ice, suspended in Lysis buffer (50 mM Tris-HCl, 300 mM NaCl, 10 mM imidazole), and ultrasonic crushing (Output 3.5, Duty 60% (TOMY UD201)) was performed on ice 20 times, 2 minutes at a time, with a cooling time of 1 minute. Centrifugal separation (10000 rpm, 30 minutes, 4° C.) of the crushed bacterial solution was performed, and the supernatant liquid was recovered.

The obtained supernatant liquid was used as an addition sample, and purified by affinity chromatography (flow velocity 15-20 ml/hour) using a column filled with Ni-NTA agarose beads equilibrated beforehand using the same buffer solution. The eluates were fractionated, and the fractions containing the target protein were verified and collected by SDS-PAGE.

After dialyzing the obtained fractions while exchanging with distilled water as the outside liquid as required for 24 to 48 hours, a white powder was obtained by freeze-drying. The yield of protein of 35 kDa was 38.8 mg.

### Example 4

<Construction of SLPF Gene>

The four oligonucleotides shown in the SEQ ID NOS: 34-37 synthesized by Asahi TechnoGlass CO., LTD., were designed. The synthesized film-like oligonucleotides were dissolved so that their concentration was 1 μg/p 1 using TE (10 mM Tris-HCl (pH 8.0), 1 mM EDTA (pH 8.0)). Double-stranded DNAs which code for the aminoacids expressed in the SEQ ID NOS: 38 and 39 were constructed by equimolar mixing of complementary strands, heat-treating at 99° C. for 30 seconds, cooling at 37° C. for 1 hour, and settling for 30 minutes. After mixing equivalent amounts of the respective double-stranded DNAs, ligation was performed at 16° C. for 1 hour using Takara Ligation Kit ver2 Solution I to prepare a double-stranded DNA coding for SLPF monomer (aminoacid sequence of SLPF: Thr Ser [Thr Gly Arg Gly Asp Ser Pro Ala Gly Gly (Gly Ala Gly Ala Gly Ser)₃ Ala Ser]ₙ, SEQ ID NO: 40 (for n=5)).

The cloning vector pUC118 was digested at 37° C. for 1 hour 30 minutes using the restriction enzyme BamHI, CIAP was added, and treatment was performed at 37° C. for 30 minutes. The reaction solution was extracted and purified with a mixture of phenol:chloroform:isoamyl alcohol in a ratio of 25:24:1 (weight ratio). Ethanol was added to the purified reaction solution, the precipitate produced was dissolved in sterilized water, and used as the vector sample.

The SLPF monomer DNA and pUC118 vector sample were mixed in a ratio of 10:1 (weight ratio), and ligation was performed at 16° C. for 1 hour using Takara Ligation Kit ver2 solution. After completion of the reaction, a transformation was performed using competent cell DH5α. The presence or absence of an insert gene was verified by color selection using X-gal, DNA sequencing was performed on the material containing the insert gene, and plasmid pUC-SLPF (1) containing the SLPF monomer DNA was obtained by verifying the sequence.

<Construction of SLPF(n)>

The two termini of SLPF monomer contain regions for recognizing the restriction enzymes Spe I and Nhe I. The projecting ends of the fragments digested by Spe I and Nhe I are all complementary and can be mutually combined. Moreover, the new sequence produced by this combination is different from both of the regions for recognizing the restriction enzymes Spe I and Nhe I, and is not digested by Spe I and Nhe I. Using this property, pUC-Link SLPF(n) was constructed by polymerizing SLPF monomer in one direction.

Competent cell DH5α was transformed using pUC-SLPF (1), and grown in 2xYT culture medium at 37° C. for 18 hours. The plasmid was extracted from the culture medium by the alkali-SDS method, and dissolved in TE. The sample was simultaneously digested by Nhe I and Spe I at 37° C. for 1 hour 30 minutes, and SLPF (1) was isolated from the plasmid. After concentrating the reaction liquid to 5 μl using MicroCon, electrophoresis was performed using 1.5% agarose gel, and the band of insert DNA was cut out. To extract DNA from the gel, the extract was concentrated to 5 μl using UltrafreeDA and then MicroCon, and this was used as the insert genetic material.

The DNA concentration in the insert genetic material was verified by electrophoresis using 1.5% agarose gel, an equivalent amount of Takara Ligation Kit ver2 Solution I to the insert gene sample was added, and ligation was performed at 16° C. for 1 hour.

After digesting pUC-Link by Nhe I, CIAP was added and alkaline phosphatase treatment was carried out. The reaction solution was extracted and purified using a mixture of phenol:chloroform:isoamyl alcohol (weight ratio 25:24:1). Ethanol was added to the purified reaction solution, the precipitate produced was dissolved in sterilized water, and this was used as the vector sample.

The DNA concentrations in the insert genetic material and vector sample were verified by electrophoresis using 1.5% agarose gel, the insert genetic material and vector sample were mixed in a ratio of 10:1, an equivalent amount of Takara Ligation Kit ver2 Solution I to the reaction mixture was added, and ligation was performed at 16° C. for 1 hour.

After the reaction was complete, transformation of competent cell DH5α was performed. It was then inoculated on a LB plate with added ampicillin, and screened. The colony produced was picked up, inoculated on 2xYT culture medium and grown at 37° C. for 18 hours. The plasmid was extracted from the culture medium by the alkali-SDS miniprep method, dissolved in TE and used as a sample. After digesting the sample simultaneously using Nhe I and Spe I, the presence or absence and size of the insert gene were verified by the electrophoresis method. Then, DNA sequencing was performed and plasmid pUC-Link SLPF (1) was obtained by verifying the sequence.

<Construction of Expression Vector pET-SLPF (5)>

pUC-SLPF (5) was digested using BamHI and Hind III. After concentrating the reaction liquid to 5 μl using MicroCon, electrophoresis was performed using a 1.5% agarose gel, and the band of insert DNA was cut out. To extract DNA from the gel, the extract was concentrated to 5 μl using UltrafreeDA and then MicroCon, and this was used as the insert genetic material.

After digesting the expression vector pET30a by BamHI and Hind III, CIAP was added and alkaline phosphatase treatment was carried out. The reaction solution was extracted and purified using a mixture of phenol:chloroform:isoamyl alcohol (weight ratio 25:24:1). Ethanol was added to the purified reaction solution, the precipitate produced was dissolved in sterilized water, and this was used as the vector sample.

The DNA concentrations in the insert genetic material and vector sample were verified by electrophoresis using 1.5% agarose gel, the insert genetic material and vector sample were mixed in a ratio of 10:1, an equivalent amount of Takara Ligation Kit ver2 Solution I to the reaction mixture was added, and ligation was performed at 16° C. for 1 hour.

Transformation of competent cell DH5α was performed using the ligation reaction solution. It was then inoculated on a LB plate with added kanamycin, and screened. The colony produced was picked up, inoculated on 2xYT culture medium and grown. The plasmid was extracted from the culture medium by the alkali-SDS method, dissolved in TE and used as a sample. After digesting the sample simultaneously using Nhe I and Spe I, the presence or absence and size of the insert gene were verified by the electrophoresis method. Then, DNA sequencing was performed and the expression vector pET-SLPF 5 was constructed by verifying the sequence.

<Expression of PET-SLPF (5)>

Host E. coli BL21 (DE3) pLysS containing the plasmid pET-SLPF (5) was grown at 37° C. for 16 hours in 1.5 ml of 2xTY (25 μg/ml kanamycin, 25 μg/ml chloramphenicol) liquid culture medium. Next, 100 μl of the culture medium was added to a test-tube containing 5 ml of 2xTY (25 μg/ml kanamycin, 25 μg/ml chloramphenicol), and cultured at 37° C. until OD₆₀₀=0.5−0.7. In this case, to induce the expression of SLPF 5, IPTG (final concentration 1 mM) was added, 100 μl of culture medium was sampled in an Eppendorf tube every other hour, and cultured for 4 hours. After performing centrifugal separation (14500 rpm, 5 minutes, 4° C.) of the sampled culture medium, the supernatant liquid was discarded, pellets were dissolved in 2× sample buffer, and heat-treated at 100° C. for 5 minutes to give a SDS-PAGE sample.

After obtaining the SDS-PAGE sample, SLPF 5 was detected by performing Western Blot using His-Tag antibody.

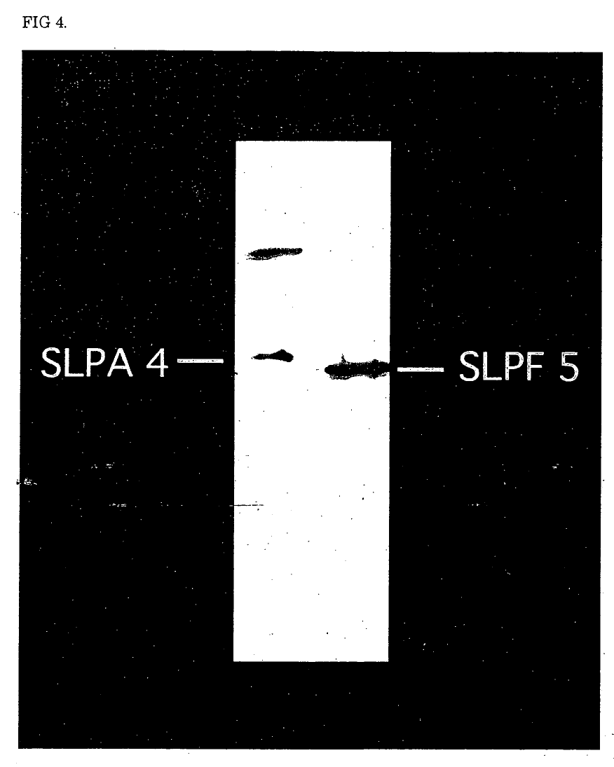

As shown in FIG. 4, with SELP 5, a band was observed at 23 kDa. From this, it was confirmed that SLPF genes were induced by IPTG addition, and mass-expressed strains could be obtained.

Host E. coli BL21 (DE3) pLysS respectively containing the plasmids pET-SLPF (5) was grown at 37° C. for 16 hours in 1.5 ml 2xTY (25 μg/ml kanamycin, 25 μg/ml chloramphenicol) liquid culture medium. Next, this culture medium was cultured at 37° C. for 16 hours in 5 ml of 2xYT (25 μg/ml kanamycin, 25 μg/ml chloramphenicol). Next, it was added to a 11 conical flask containing 250 ml of 2xYT (25 μg/ml kanamycin, 25 μg/ml chloramphenicol) liquid culture medium, and cultured at 37° C. until OD₆₀₀=0.5−0.7. In this case, IPTG (final concentration 0.2 mM) was added to induce expression of the protein, and a bacteria was obtained by culturing for a further 4 hours and collecting (8500 rpm, 30 minutes, 4° C.). The obtained bacteria was stored at −20° C.

The aforesaid bacteria stored at −20° C. was slowly thawed on ice, suspended in Lysis buffer (50 mM Tris-HCl, 300 mM NaCl, 10 mM imidazole), and ultrasonic crushing (Output 3.5, Duty 60% (TOMY UD201)) was performed on ice 20 times, 2 minutes at a time, with a cooling time of 1 minute. Centrifugal separation (10000 rpm, 30 minutes, 4° C.) of the crushed bacterial solution was performed, and the supernatant liquid was recovered.

The obtained supernatant liquid was used as an addition sample, and purified by affinity chromatography (flow velocity 15-20 ml/hour) using a column filled with Ni-NTA agarose beads equilibrated beforehand using the same buffer solution. The eluates were fractionated, and the fractions containing the target protein were verified and collected by SDS-PAGE.

After dialyzing the obtained fractions while exchanging with distilled water as the outside liquid as required for 24 to 48 hours, a white powder was obtained by freeze-drying. The yield of protein of 23 kDa was 38.8 mg/L.

What is claimed is:

1. A method of producing silk or silk-like protein wherein silk or a silk-like polymer comprising at least one protein selected from among domestic silkworm fibroin, wild silkworm fibroin, elastin and fibronectin, and essentially comprising the aforesaid domesticated silkworm fibroin or silkworm fibroin is designed, and the minimum unit of the silk or the thus designed polymer is synthesized, the polymer of the minimum unit thus synthesized is integrated into at least one expression vector selected from among expression vectors containing T7 promoter, the expression vector is integrated into E. coli BL21 (DE3) pLysS or BLR(DE3) pLysS, and the E. coli is grown in a medium selected from among composite media.

2. The method of producing silk or gene-recombinant silk-like protein according to claim 1, wherein the E. coli is grown at a temperature 2-7° C. below the optimum proliferation temperature of E. coli.

3. The method of producing silk or gene-recombinant silk-like protein according to claim 1, wherein the expression vector is selected from among expression vectors containing T71ac promoter.

4. The method of producing silk or gene-recombinant silk-like protein according to claim 3, wherein the expression vector is pET30a.

5. The method of producing silk or gene-recombinant silk-like protein according to claim 1, wherein the culture medium is a TB culture medium.


SEQUENCE LISTING (40 sequences)

SEQ ID NO: 1 - protein, 5 aa, Artificial Sequence
  Description of Artificial Sequence Synthetic peptide
VPGVG

SEQ ID NO: 2 - protein, 4 aa, Artificial Sequence
  Description of Artificial Sequence Synthetic peptide
RGDS

SEQ ID NO: 3 - protein, 8 aa, Artificial Sequence
  Description of Artificial Sequence Synthetic peptide
TGRGDSPA

SEQ ID NO: 4 - protein, 18 aa, Artificial Sequence
  Description of Artificial Sequence Synthetic peptide
GAGSGAGAGSGAGAGSGA

SEQ ID NO: 5 - protein, 20 aa, Artificial Sequence
  Description of Artificial Sequence Synthetic peptide
GGAGSGYGGGYGHGYGSDGG

SEQ ID NO: 6 - protein, 6 aa, Artificial Sequence
  Description of Artificial Sequence Synthetic peptide
GVGAGY

SEQ ID NO: 7 - protein, 6 aa, Artificial Sequence
  Description of Artificial Sequence Synthetic peptide
GAGAGY

SEQ ID NO: 8 - protein, 6 aa, Artificial Sequence
  Description of Artificial Sequence Synthetic peptide
GAGVGY

SEQ ID NO: 9 - protein, 18 aa, Artificial Sequence
  Description of Artificial Sequence Synthetic peptide
AAAAAAAAAAAAAAAAAA

SEQ ID NO: 10 - protein, 10 aa, Artificial Sequence
  Description of Artificial Sequence Synthetic peptide
GVPGVGVPGV

SEQ ID NO: 11 - protein, 8 aa, Artificial Sequence
  Description of Artificial Sequence Synthetic peptide
TGRGDSPA

SEQ ID NO: 12 - DNA, 65 bp, Artificial Sequence
  Description of Artificial Sequence Synthetic nucleotide sequence
gatccactagtggtggtgctggtagcggctatggcggcggctacggtcatggttatggtagcgat

SEQ ID NO: 13 - DNA, 67 bp, Artificial Sequence
  Description of Artificial Sequence Synthetic nucleotide sequence
acccccatcgctaccataaccatgaccgtagccgccgccatagccgctaccagcaccaccactagtg

SEQ ID NO: 14 - DNA, 67 bp, Artificial Sequence
  Description of Artificial Sequence Synthetic nucleotide sequence
gggggtggtgcaggtgctggctccggtgccggcgcggggagcggggcaggcgcaggttctgctagcg

SEQ ID NO: 15 - DNA, 65 bp, Artificial Sequence
  Description of Artificial Sequence Synthetic nucleotide sequence
gatccgctagcagaacctgcgcctgccccgctccccgcgccggcaccggagccagcacctgcacc

SEQ ID NO: 16 - protein, 22 aa, Artificial Sequence
  Description of Artificial Sequence Synthetic peptide
TSGGAGSGYGGGYGHGYGSDGG

SEQ ID NO: 17 - protein, 22 aa, Artificial Sequence
  Description of Artificial Sequence Synthetic peptide
GGGAGAGSGAGAGSGAGAGSAS

SEQ ID NO: 18 - protein, 242 aa, Artificial Sequence
  Description of Artificial Sequence Synthetic peptide
TSGGAGSGYGGGYGHGYGSDGGGAGAGSGAGAGSGAGAGSASGGAGSGYGGGYGHGYGSDGGGAGAGSGAGAGSGAGAGSASGGAGSGYGGGYGHGYGSDGGGAGAGSGAGAGSGAGAGSASGGAGSGYGGGYGHGYGSDGGGAGAGSGAGAGSGAGAGSASGGAGSGYGGGYGHGYGSDGGGAGAGSGAGAGSGAGAGSASGGAGSGYGGGYGHGYGSDGGGAGAGSGAGAGSGAGAGSAS

SEQ ID NO: 19 - DNA, 30 bp, Artificial Sequence
  Description of Artificial Sequence Synthetic nucleotide sequence
ctagaatgactagtgggcccgctagcatgt

SEQ ID NO: 20 - DNA, 75 bp, Artificial Sequence
  Description of Artificial Sequence Synthetic nucleotide sequence
gatccatggctagcgctgctgcggcagcagcggctgctgcggcagcagcggctgctgcggcagcagcgactagtg

SEQ ID NO: 21 - DNA, 75 bp, Artificial Sequence
  Description of Artificial Sequence Synthetic nucleotide sequence
gatccactagtcgctgctgccgcagcagccgctgctgccgcagcagccgctgctgccgcagcagcgctagccatg

SEQ ID NO: 22 - DNA, 111 bp, Artificial Sequence
  Description of Artificial Sequence Synthetic nucleotide sequence
gatccatggctagcggcgtcggtgcaggctatggtgctggcgcagggtacggtgttggcgccggttatggcgcgggagtgggttatggcgcaggtgctggctacactagtg

SEQ ID NO: 23 - DNA, 111 bp, Artificial Sequence
  Description of Artificial Sequence Synthetic nucleotide sequence
gatccactagtgtagccagcacctgcgccataacccactcccgcgccataaccggcgccaacaccgtaccctgcgccagcaccatagcctgcaccgacgccgctagccatg

SEQ ID NO: 24 - protein, 20 aa, Artificial Sequence
  Description of Artificial Sequence Synthetic peptide
ASAAAAAAAAAAAAAAAAAA

SEQ ID NO: 25 - protein, 34 aa, Artificial Sequence
  Description of Artificial Sequence Synthetic peptide
TSGVGAGYGAGAGYGVGAGYGAGVGYGAGAGYTS

SEQ ID NO: 26 - protein, 210 aa, Artificial Sequence
  Description of Artificial Sequence Synthetic peptide
ASAAAAAAAAAAAAAAAAAATSGVGAGYGAGAGYGVGAGYGAGVGYGAGAGYTSAAAAAAAAAAAAAAAAAATSGVGAGYGAGAGYGVGAGYGAGVGYGAGAGYTSAAAAAAAAAAAAAAAAAATSGVGAGYGAGAGYGVGAGYGAGVGYGAGAGYTSAAAAAAAAAAAAAAAAAATSGVGAGYGAGAGYGVGAGYGAGVGYGAGAGYTS

SEQ ID NO: 27 - DNA, 41 bp, Artificial Sequence
  Description of Artificial Sequence Synthetic nucleotide sequence
aattcactagtggtgtaccaggtgttggcgttccgggtgtg

SEQ ID NO: 28 - DNA, 43 bp, Artificial Sequence
  Description of Artificial Sequence Synthetic nucleotide sequence
accccccacacccggaacgccaacacctggtacaccactagtg

SEQ ID NO: 29 - DNA, 67 bp, Artificial Sequence; identical to SEQ ID NO: 14
  Description of Artificial Sequence Synthetic nucleotide sequence

SEQ ID NO: 30 - DNA, 65 bp, Artificial Sequence; identical to SEQ ID NO: 15
  Description of Artificial Sequence Synthetic nucleotide sequence

SEQ ID NO: 31 - protein, 14 aa, Artificial Sequence
  Description of Artificial Sequence Synthetic peptide
TSGVPGVGVPGVGG

SEQ ID NO: 32 - protein, 22 aa, Artificial Sequence
  Description of Artificial Sequence Synthetic peptide
GGGAGAGSGAGAGSGAGAGSAS

SEQ ID NO: 33 - protein, 258 aa, Artificial Sequence
  Description of Artificial Sequence Synthetic peptide
TSGVPGVGVPGVGGGAGAGSGAGAGSGAGAGSASGVPGVGVPGVGGGAGAGSGAGAGSGAGAGSASGVPGVGVPGVGGGAGAGSGAGAGSGAGAGSASGVPGVGVPGVGGGAGAGSGAGAGSGAGAGSASGVPGVGVPGVGGGAGAGSGAGAGSGAGAGSASGVPGVGVPGVGGGAGAGSGAGAGSGAGAGSASGVPGVGVPGVGGGAGAGSGAGAGSGAGAGSASGVPGVGVPGVGGGAGAGSGAGAGSGAGAGSAS

SEQ ID NO: 34 - DNA, 39 bp, Artificial Sequence
  Description of Artificial Sequence Synthetic nucleotide sequence
aattcataaactagtactggccgtggtgattctccggct

SEQ ID NO: 35 - DNA, 41 bp, Artificial Sequence
  Description of Artificial Sequence Synthetic nucleotide sequence
acccccagccggagaatcaccacggccagtactagtttatg

SEQ ID NO: 36 - DNA, 67 bp, Artificial Sequence; identical to SEQ ID NO: 14
  Description of Artificial Sequence Synthetic nucleotide sequence

SEQ ID NO: 37 - DNA, 65 bp, Artificial Sequence; identical to SEQ ID NO: 15
  Description of Artificial Sequence Synthetic nucleotide sequence

SEQ ID NO: 38 - protein, 12 aa, Artificial Sequence
  Description of Artificial Sequence Synthetic peptide
TSTGRGDSPAGG

SEQ ID NO: 39 - protein, 22 aa, Artificial Sequence
  Description of Artificial Sequence Synthetic peptide
GGGAGAGSGAGAGSGAGAGSAS

SEQ ID NO: 40 - protein, 152 aa, Artificial Sequence
  Description of Artificial Sequence Synthetic peptide
TSTGRGDSPAGGGAGAGSGAGAGSGAGAGSASTGRGDSPAGGGAGAGSGAGAGSGAGAGSASTGRGDSPAGGGAGAGSGAGAGSGAGAGSASTGRGDSPAGGGAGAGSGAGAGSGAGAGSASTGRGDSPAGGGAGAGSGAGAGSGAGAGSAS